(19) United States
(12) Patent Application Publication
(10) Pub. No.: US 2005/0177245 A1
(43) Pub. Date: Aug. 11, 2005

(54) Absorbable orthopedic implants

(76) Inventors: Neil C. Leatherbury, San Antonio, TX (US); Fred B. Dinger III, San Antonio, TX (US); Jeffrey S. Wrana, San Antonio, TX (US); David Caborn, Goshen, KY (US)

Correspondence Address: GREENLEE WINNER AND SULLIVAN P C, 4875 PEARL EAST CIRCLE, SUITE 200, BOULDER, CO, 80301, US

(21) Appl. No.: 11/052,626

(22) Filed: Feb. 7, 2005

Related U.S. Application Data
(60) Provisional application No. 60/542,640, filed on Feb. 5, 2004.

(51) Int. Cl. A61F 2/28; A61F 2/44

(52) U.S. Cl. 623/23.5; 623/23.51; 623/17.11

(57) ABSTRACT

The invention provides orthopedic implants which are at least partially absorbable. The implants of the invention may include a biocompatible material in the form of a ring, which may be used in combination with a second, more porous, absorbable material. This second material may be a continuous body or discontinuous. The implant may also include a first material connected to a full or partial wedge of a second material, the wedge being connected to the inner surface of the more dense material. Suitable materials for the first and second materials include, but are not limited to, resorbable polymer composites. The implants of the invention may also include plates for anchoring of the implant.

### CROSS-REFERENCE TO RELATED APPLICATIONS

This application claims the benefit of U.S. Provisional Application 60/542,640, filed Feb. 5, 2004, which is hereby incorporated by reference to the extent not inconsistent with the disclosure herein.

### BACKGROUND OF THE INVENTION

This invention is in the field of orthopedic implants, in particular, implants which are made at least in part of absorbable material.

The patent literature describes a variety of tissue implant materials and devices having two regions of differing composition and/or microstructure.

U.S. Pat. Nos. 6,149,688 and 6,607,557, to Brosnahan et al., describe an artificial bone graft implant having two basic portions, each composed of a biocompatible microporous material. The core of the implant is formed of a highly porous composition and the shell of a low porosity dense composition. An implant formed of a unitary structure having a gradient of pore sizes is also described. Specific implant materials mentioned include biocompatible metallics, ceramics, polymers, and composite materials consisting of phosphate(s), bioactive glass(es) and bioresorbable polymer(s).

U.S. Pat. No. 5,769,897, to Härle, describes an artificial bone material which has a strength sustaining first component and a biointegration promoting second component. The first and second materials can be selected from a group including bioceramic materials, carbon ceramics, aluminum oxide ceramics, glass ceramics, tricalcium phosphate ceramics, tetracalciumphosphate ceramics, hydroxylapatite, polyvinylmethacrylate, titanium, implantation alloys, and biocompatible fiber materials.

U.S. Pat. No. 5,152,791, to Hakamatsuka et al., describes a prosthetic artificial bone having a double-layered structure obtained by molding a porous portion having a porosity from 40 to 90% and a dense portion having a porosity not more than 50% into an integral body. The implant material is a ceramic or glass containing calcium and phosphorus.

U.S. Pat. No. 5,607,474, to Athanasiou et al., describes a multi-phase bioerodible polymeric implant/carrier. U.S. Pat. No. 6,264,701 to Brekke describes bioresorbable polymer devices having a first region with an internal three-dimensional architecture to approximate the histologic pattern of a first tissue; and a second region having an internal three-dimensional architecture to approximate the histologic pattern of a second tissue. U.S. Pat. No. 6,365,149, to Vyakarnam et al., describes gradients in composition and/or microstructure in porous resorbable polymer forms. U.S. Pat. No. 6,454,811, to Sherwood et al., describes use of gradients in materials and/or macroarchitecture and/or microstructure and/or mechanical properties in synthetic polymeric materials.

U.S. Pat. No. 4,863,472, to Törmälä et al, describes a bone graft implant having bone graft powder located inside and/or below a supporting structure. The supporting structure is manufactured at least partially of a resorbable polymer, copolymer or polymer blend. The supporting structure also includes porosity which allows the surrounding tissues to grow through the supporting structure but which prevents the migration of the bone graft powder through the pores outside the supporting structure.

Further, the patent literature describes implants which contain cavities or spaces which can be filled with material to induce bone growth.

U.S. Pat. No. 6,548,002, to Gresser et al., describes a spinal wedge incorporating peripheral and/or central voids which can be filled with grafting material for facilitating bony development and/or spinal fusion. The wedge can be made of a biodegradable, biocompatible polymer which may include a buffer.

U.S. Pat. No. 6,652,073 and U.S. Published Patent Application No. 2003/1095632, both to Foley et al., describe implants having a cavity in which bone growth material is placed. U.S. Pat. No. 6,652,073 describes an implant body of bone. U.S. Published Application 2003/1095632 lists titanium, composite materials, including carbon composites, and surgical stainless steel as examples of suitable implant body materials. For spinal implants, a variety of methods have been described for securing the implant. U.S. Pat. No. 6,576,017, to Foley et al., U.S. Pat. No. 6,562,073, to Foley, U.S. Pat. No. 6,461,359, to Tribus et al, and U.S. Pat. No. 5,645,599, to Samani et al., describe devices with an intervertebral body and flange-like structures. The flange-like structures can be attached to vertebrae. U.S. Pat. No. 5,306,309 to Wagner et al. describes a spinal disk implant in which the intervertebral body has an engagement region which has one or more three-dimensional features extending above the general level of the transverse faces. The engagement features are intended to sink into the cancellous bone as load is applied.

### SUMMARY OF THE INVENTION

The invention provides absorbable orthopedic implants. The implants of the invention are useful for applications including, but not limited to, osteotemies, spinal interbody fusion, long bone lengthening, and trauma reconstruction.

In an embodiment, the invention provides an implant comprising a ring of biocompatible material. The ring may be made of an absorbable biocompatible material. The ring may be used in combination with a second, more porous, absorbable material. This second material may be a continuous body or composed of multiple pieces (e.g. granules or chunks). The ring may be connected to one or more plates which allow attachment of the ring to neighboring bone. For example, in a spinal implant, anterior plates allow attachment of the implant to neighboring vertebrae.

In another embodiment, the invention provides an implant comprising a first material, not in the form of a ring, in combination with a full or partial wedge of a second, more porous, absorbable material. The first material may be connected to one or more plates which allow attachment of the implant to neighboring bone.

Suitable materials for the implants of the invention include, but are not limited to, absorbable polymer composites. One suitable absorbable material is a fully dense composite of an absorbable material with ceramic or mineral particles. Another suitable absorbable material is a porous composite of an absorbable polymer, ceramic or mineral particles, fibers, and a surfactant. Inclusion of ceramic or mineral particles can provide a buffering affect, increase osteoconductivity, and increase the mechanical strength of the composite.

### BRIEF DESCRIPTION OF THE FIGURES

FIG. 1A is a perspective view of a wedge-shaped implant of the invention.

FIG. 1B is an exploded view of the implant of FIG. 1A.

FIG. 1C is a side view of the implant of FIG. 1A, illustrating the wedge angles (θ₁, θ₂) of the first and second regions of the implant.

FIG. 2A is a perspective view of a wedge-shaped implant with an attached anterior plate.

FIG. 2B is a side view of the implant of FIG. 2A, illustrating the wedge angles θ₁, θ₂ and the angle θ₃ between the plate and the midplane of the wedge.

FIG. 3A is a side view of another implant of the invention, where the angle θ₃ between the midplane of the wedge and the plane of the plate is less than ninety degrees.

FIG. 3B is a front view of another implant of the invention, where the angle θ₄ between the midplane of the wedge and the midplane of the plate is less than ninety degrees.

FIG. 4 is a perspective view of a tissue spacer comprising a less porous load bearing outer ring and a more porous inner core.

FIG. 5 is a perspective view of a tissue spacer with a load-bearing ring connected to an anterior plate.

FIG. 6A is a front view of an implant comprising a wedge attached to an anterior plate, illustrating distances E and F between the ends of the anterior plate.

FIG. 6B is a front view of the implant of FIG. 6A, illustrating distances A, B, and C.

FIG. 6C is a side view of the implant in FIGS. 6A and 6B, illustrating distance D.

FIG. 7A is a side view of a tissue spacer comprising a load-bearing ring attached to an anterior plate. The thickness of the spacer, tₛ, is illustrated.

FIG. 7B is a top view of the implant of FIG. 7A, illustrating the thickness, length, and width of the load-bearing ring.

FIG. 7C is a front view of the tissue spacer of FIG. 7A, illustrating the distance H.

### DETAILED DESCRIPTION OF THE INVENTION

The invention provides orthopedic implants comprising absorbable material. The implants of the invention comprise a tissue spacer which comprises absorbable material and, optionally, one or more plates for attachment to tissue. The tissue spacer may be wholly absorbable or may contain some nonabsorbable components. Nonabsorbable components of the tissue spacer can include, but are not limited to, load-bearing portions of the tissue spacer or reinforcement materials such as fibers. The anterior plates may be absorbable or nonabsorbable. The terms “biodegradable” and “absorbable” are used interchangeably to mean capable of breaking down over time, either inside a patient's body, or when used with cells to grow tissue outside the body. When placed inside a patient's body, the absorbable portions of the implants of the invention will degrade over time and be removed by the body's natural processes.

In an embodiment, the invention provides an orthopedic implant comprising
a tissue spacer having a superior and an inferior surface, the tissue spacer comprising
   a first region having a superior surface which forms part of the superior surface of the spacer, an inferior surface which forms part of the inferior surface of the spacer, an inner surface and an outer surface; and
   a second region in the form of a full or partial wedge, the second region having a superior surface which forms part of the superior surface of the spacer, an inferior surface which forms part of the inferior surface of the spacer, and an anterior surface connected to the inner surface of the first region;
wherein the second region is more porous than the first region and comprises absorbable material and the first region is not in the form of a complete ring.

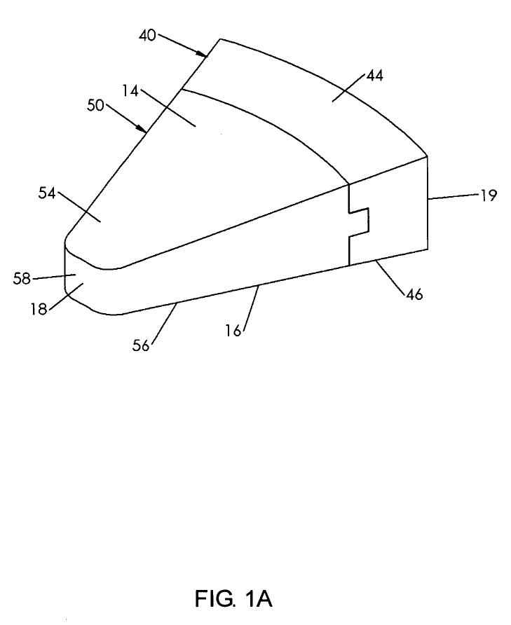

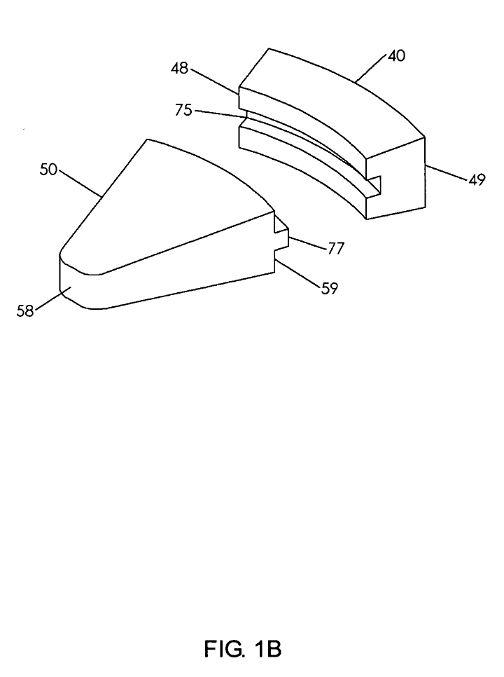

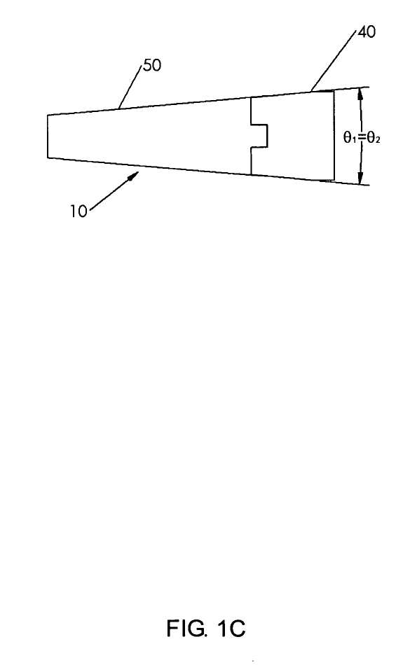

FIGS. 1A-1C illustrate an embodiment where the tissue spacer (10) comprises a porous partial wedge (50) connected to the first region (40). In this embodiment, the first region (40) does not form a complete ring. The tissue spacer has a superior surface (14), an inferior surface (16), a posterior surface (18) and an anterior surface (19). The first region (40) has a superior surface (44), an inferior surface (46), an inner surface (48) and an outer surface (49). In FIG. 1A, outer surface (49) of the first region (40) forms the anterior surface (19) of the tissue spacer. The partial wedge (50) has a superior surface (54), an inferior surface (56), a posterior surface (58) and an anterior surface (59). In FIG. 1A, the posterior surface (58) of the partial wedge forms the posterior surface (18) of the spacer. FIGS. 1A and 1B illustrate connection of the inner surface (48) of the first region (40) to the anterior surface (59) of the partial wedge (50) by a slot (75) and bar (77) connection. More generally, the first region may be mechanically attached to the porous wedge by a slot and bar connection, a dove tail connection, a hole and post connection, a threaded post, various snap fits or other forms of mechanical connections known to those skilled in the art.

As used herein, a full wedge is the shape formed by two inclined planes that merge to form an edge. Partial wedge shapes suitable for use with the invention exclude at least the edge or tip of a full wedge shape, and may exclude a larger portion of the tip end of a full wedge. The angle of a wedge or partial wedge is the angle of inclination between the planes forming the superior and inferior surfaces of the wedge or partial wedge. The angle of inclination, θ₁ can be between about 5 degrees and about 20 degrees. In different embodiments, the angle θ₁ is 7.5 degrees, 10 degrees, 12.5 degrees, or 15 degrees.

As used herein, a ring may be in the form of a circle, an oval, a rectangle, or another shape forming a closed curve. In the embodiment shown in FIGS. 1A-1C, the first region (40) takes the form of an arc. The shape of the first region in FIGS. 1A-1C can be regarded as a section of a curved ring, the ring section having an arc length less than that of a full ring. In different embodiments, the first region may take the form of a section of a rectangular ring, including U-shapes and bars. The shape of the first region may be selected based on the intended implant location. In the embodiment shown in FIGS. 1A-1C, the first region (40) contacts porous wedge (50) only at the anterior portion of the porous wedge (50) and does not “wrap around” the sides of the wedge.

In general, the planes forming the superior and inferior surfaces of the first material need not be parallel and can have an angle of inclination θ₂ between them. Typically, as shown in FIG. 1C, the angle of inclination θ₂ is equal to angle of inclination θ₁.

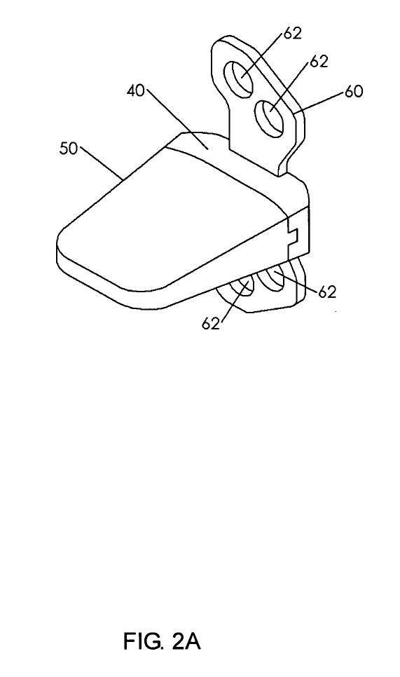

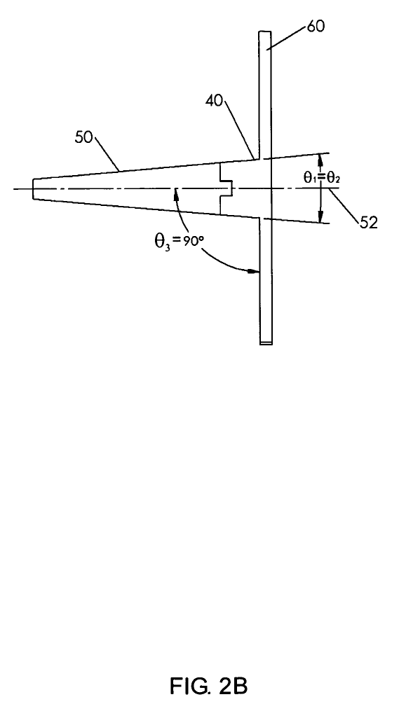

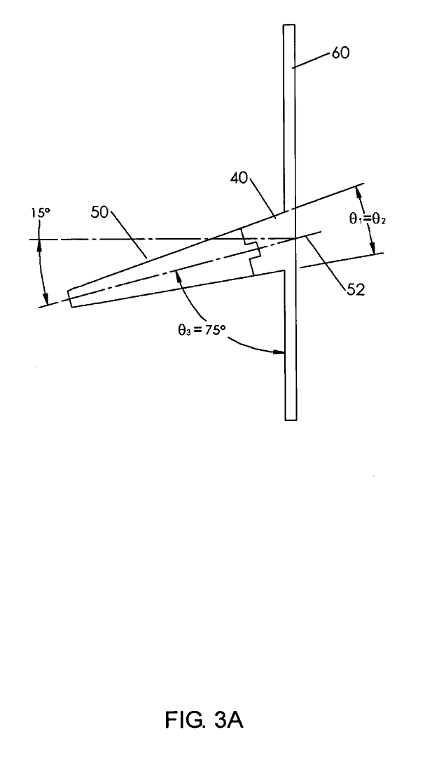

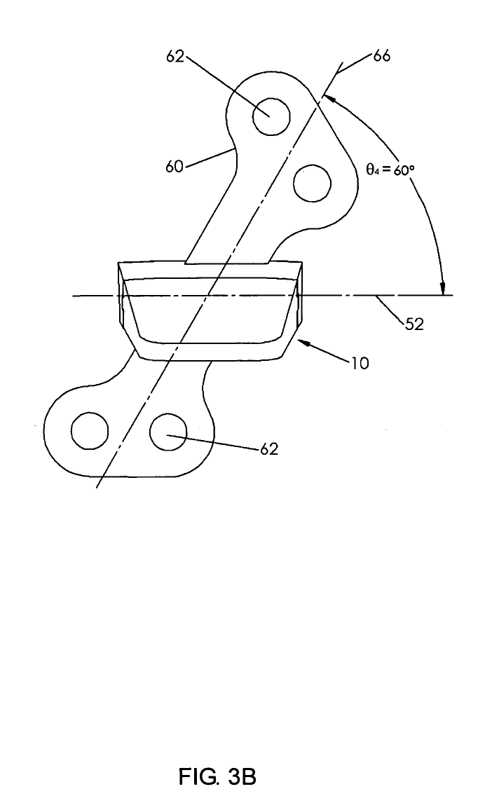

A tissue spacer comprising a first region connected to a full or partial porous wedge can also be connected to one or more anterior plates (60) as shown in FIGS. 2A and 2B. FIG. 2A shows holes (62) for attachment of the anterior plate to a bone. The tissue spacer can be attached to the anterior plate so that the midplane (52) dividing the full or partial wedge into superior and inferior portions forms an angle θ₃ of 90 degrees with respect to the plate (60), as shown in FIG. 2B. As shown in FIG. 3A, the tissue spacer can also be attached to the anterior plate so that the midplane (52) of the full or partial wedge forms an angle θ₃ other than 90 degrees with respect to the plate (as measured at the junction of the tissue spacer with the anterior plate through the inferior portion of the tissue spacer). The angle θ₃ can be between about 55 degrees and about 90 degrees. In an embodiment, θ₃ is between about 60 and about 75 degrees. FIG. 3A illustrates an embodiment in which the midplane (52) of a partial wedge tissue spacer forms an angle θ₃ of about 75 degrees with respect to the anterior plate.

In addition, as shown in FIG. 3B, the anterior surface of the tissue spacer can be attached to the anterior plate so that midplane (52) forms an angle θ₄ with respect to the midplane (66) dividing the anterior plate into left and right portions. Angle θ₄ can be measured through either the left or the right portions of the anterior plate. FIG. 3B illustrates an embodiment where the angle θ₄ between a partial wedge midplane (52) and an anterior plate midplane (66) is about 60 degrees. In an embodiment, the angle θ₄ is between about 30 degrees and about 90 degrees.

The implant comprising a tissue spacer comprising a first region connected to a porous full or partial wedge can be useful in spinal applications as well as for osteotomies, long bone lengthening, and trauma reconstruction. An osteotomy is a surgical procedure necessary to correct a patient's bone alignment. In an osteotomy, the bone is transected or cut to realign the bone ends.

The first region preferably has an initial Young's Modulus between about 1.0 GPa and about 30 GPa and a compressive strength between about 10 MPa and about 500 MPa. In an embodiment the initial Young's Modulus is between about 10 GPa and about 30 GPa. At between six to nine months after implantation, the first region preferably retains between about 70% to 90% of its initial strength. In an embodiment, the first region preferably retains about 80% of its initial strength. The full or partial wedge preferably has an initial Young's Modulus between about 0.5 Gpa and about 5 GPa. In an embodiment, the initial Young's Modulus is between about 1 GPa and about 5 GPa and a compressive strength between zero MPa and about 30 MPa. In an embodiment, the first region and/or full or partial wedge has mechanical properties matching those of bone tissue into which it is to be inserted. These mechanical properties include a Young's modulus of about 15 GPa for cortical bone and a Young's modulus of about 500 MPa for cancellous bone.

In different embodiments, the porosity of the first region is between zero and about 30%, or between zero and about 15%. In an embodiment, the first region is substantially nonporous, having porosity less than about 5%.

In an embodiment, the first region of the tissue spacer is formed of a substantially nonporous (fully dense) absorbable material comprising absorbable polymer, an optional ceramic or mineral component such as beta-tricalcium phosphate and an optional buffering component such as calcium carbonate. The absorbable polymer selected is soluble or at least swellable in a solvent and is able to degrade in-vivo without producing toxic side products. Typical polymers are selected from the family of poly-lactide, poly-glycolide, poly-caprolactone, poly-dioxanone, poly-trimethylene carbonate, and their co-polymers; however any absorbable polymer can be used. Polymers known to the art for producing biodegradable implant materials include polyglycolide (PGA), copolymers of glycolide such as glycolide/L-lactide copolymers (PGA/PLLA), glycolide/trimethylene carbonate copolymers (PGA/TMC); polylactides (PLA), stereocopolymers of PLA such as poly-L-lactide (PLLA), Poly-DL-lactide (PDLLA), L-lactide/DL-lactide copolymers; copolymers of PLA such as lactide/tetramethylglycolide copolymers, lactide/trimethylene carbonate copolymers, lactide/.delta.-valerolactone copolymers, lactide.epsilon.-caprolactone copolymers, polydepsipeptides, PLA/polyethylene oxide copolymers, unsymmetrically 3,6-substituted poly-1,4-dioxane-2,5-diones; polyhydroxyalkanate polymers including poly-beta-hydroxybutyrate (PHBA), PHBA/beta-hydroxyvalerate copolymers (PHBA/HVA), and poly-beta-hydroxypropionate (PHPA), poly-p-dioxanone (PDS), poly-delta-valerolatone, poly-epsilon-caprolactone, methylmethacrylate-N-vinyl pyrrolidone copolymers, polyesteramides, polyesters of oxalic acid, polydihydropyrans, polyalkyl-2-cyanoacrylates, polyurethanes (PU), polyvinyl alcohol (PVA), polypeptides, poly-beta-maleic acid (PMLA), and poly-beta-alkanoic acids. The polymer can be chosen, as is known to the art, to have a selected degradation period. For intervertebral spacers the degradation period is preferably up to about 4 years, or between about 6 weeks and about 2 years, or between about 12 weeks and about 1 year. For osteotomy wedges, the degradation period is preferably up to about 2 years, or between about 3 weeks and about 1 year, or between about 6 weeks and about 9 months.

The ceramic or mineral component of the material adds both mechanical reinforcement and biological activity to the material. The ceramic (or mineral) component is chosen from calcium sulfate (hemi- or di-hydrate form), salts of calcium phosphate such as tricalcium phosphate or hydroxyGPatite, various compositions of Bioglass®, and blends or combinations of these materials. In an embodiment, more than one mineral component is included in the composite. Particles can range in size from sub-micron to up to 1 mm, depending on the desired role of the component chosen. The volume fraction of ceramic particles can range from about 1% to about 40%, from about 5% to about 30%, or from about 10% to about 20%.

Incorporation of calcium-containing minerals can help buffer the degradation of biodegradable polymers. Other useful buffering compounds include compounds as disclosed in U.S. Pat. No. 5,741,321 to Agrawal et al., hereby incorporated by reference. Volume fractions of the buffering compound are from about 1% to about 40%, from about 5% to about 30%, or from about 10% to about 20%. Particle sizes for the buffering compound are less than about 2 mm or less than about 1 mm.

Either absorbable or nonabsorbable fibers can also be added to the material to provide additional reinforcement as is known to those skilled in the art. The fibers may be aligned or have a random orientation.

Fully dense portions of the tissue spacer can be fabricated by injection molding, machining, or other methods as known in the art.

The first region may also be made of nonabsorbable biocompatible material such as a metal, a plastic, or a ceramic. Nonabsorbable materials suitable for use in implants are known to those skilled in the art.

In an embodiment, the porosity of the full or partial wedge is between about 50% and about 90%. In different embodiments, the porosity of the full or partial wedge is greater than about 50% or greater than about 70%. Preferably, the full or partial wedge is sufficiently porous to allow for bony ingrowth. In an embodiment, the average pore size of the full or partial wedge between about 10 microns and about 2000 microns, between about 50 microns and about 900 microns and about 100 microns to about 600 microns. The more porous portion of the tissue spacer can be capable of soaking up fluids such as blood or bone marrow and therefore can be loaded with bioactive agents, drugs or pharmaceuticals. Both autologous and bioactive agents can be used with the tissue spacer of the invention. Autologous bioactive agents include, but are not limited to, concentrated blood, such as Platelet-Rich Plasma (PRP) and Autologous Growth Factor (AGF), and the patient's own bone marrow. Synthetic bioactive agents, include, but are not limited to, bone morphogenic proteins (e.g. BMP-2 growth factors (VEGF, FGF, TGF-b, PDGF, IGF) or synthetic or analogous versions of these peptides).

The implant may also be seeded with cells of the type whose ingrowth is desired. Osteoblasts and osteocytes are bone-forming cells which could be adsorbed onto the porous portion of the device. Mesenchymal stem cells, bone marrow cells, or other precursor cells which have the potential to differentiate into bone-forming cells may also be used.

The implant material of this invention can also be preseeded with autologous or allogenic tissue. The autologous or allogenic tissue may be minced or particulated. In an embodiment, the tissue is dermal tissue, cartilage, ligament, tendon, or bone. These allogenic tissues can be processed to preserve their biological structures and compositions, but to remove cells which may cause an immune response. Similarly, autologous tissues can be utilized and processed as described for allografts.

In an embodiment, the porous full or partial wedge comprises up to four main components: 1) an absorbable polymer, 2) a ceramic, 3) fibers, and 4) a surfactant. The device can be prepared with only the first two components; however additional performance properties can be achieved with addition of the third and fourth components. Porous materials made with these components provide a porous polymeric scaffold, incorporate a high level of biologically active or biologically compatible ceramic or mineral, and provide a high level of toughness and strength. When the material includes surfactant, the porous material becomes more wettable, overcoming some of the limitations of the intrinsically hydrophobic material. Table 1 lists typical percentages of each of these four components. Table 2 lists typical physical properties of the formulations in Table 1.

The absorbable polymer forms the core component of the porous portion of the tissue spacer and is needed for formation of the porous structure of the implant material. The polymer selected is soluble or at least swellable in a solvent and is able to degrade in-vivo without producing toxic side products. Typical polymers are selected from the family of poly-lactide, poly-glycolide, poly-caprolactone, poly-dioxanone, poly-trimethylene carbonate, and their co-polymers; however any absorbable polymer or combinations of absorbable polymers can be used. The polymer has a molecular weight sufficient to form a viscous solution when dissolved in a volatile solvent, and ideally precipitates to form a soft gel upon addition of a non-solvent. The polymer can be selected as is known to the art to have a desired degradation period. For a full or partial wedge, the degradation period is preferably up to about 2 years, or between about 3 weeks and about 1 year, or between about 6 weeks and about 9 months.

The ceramic component of the material adds both mechanical reinforcement and biological activity to the material. The ceramic (or mineral) component is chosen from calcium sulfate (hemi- or di-hydrate form), salts of calcium phosphate such as tricalcium phosphate or hydroxyapatite, various compositions of Bioglass®, and blends or combinations of these materials. Particles can range in size from sub-micron to up to 1 mm, depending on the desired role of the component chosen. For example, a highly-reinforced composite material can be prepared by incorporating nano-particles of hydroxyapatite. Alternatively, large particles of calcium sulfate (>100 μm) can be incorporated which will dissolve in 4 to 6 weeks, increasing the overall porosity of the material and stimulating bone formation. Incorporation of calcium containing minerals can also help buffer the degradation of biodegradable polymers to avoid acidic breakdown products. The ceramic component can also take the shape of elongated particles or fibers to provide enhanced mechanical properties.

Addition of fibers to the composite can increase both the toughness and strength of the material, as is well known to the art. Fibers suitable for use with the invention include both absorbable and nonabsorbable fibers. Preferential alignment of fibers in a porous material can produce anisotropic behavior as described in U.S. Pat. No. 6,511,511, where the strength is increased when the load is applied parallel to the primary orientation of the fibers. In the present invention, up to 30% by mass of the material can be comprised of fibers. Preferred polymeric fiber materials can be selected from the family of poly-lactide, poly-glycolide, poly-caprolactone, poly-dioxanone, poly-trimethylene carbonate, and their co-polymers; however any absorbable polymer could be used. Polysaccharide-based fibers can be chosen from cellulose, chitosan, dextran, and others, either functionalized or not. Non-polymeric fibers can be selected from spun glass fibers (e.g. Bioglass®, calcium phosphate glass, soda glass) or other ceramic materials, carbon fibers, and metal fibers.

The optional addition of a bio-compatible surfactant can improve the surface wettability of the porous construct. This can improve the ability of blood, body fluids, and cells to penetrate large distances into the center of an implant by increasing the capillary action. Examples of bio-compatible surfactants are poly-ethylene oxides (PEO's), poly-propylene oxides (PPO's), block copolymers of PEO and PPO (such as Pluronic surfactants by BASF), polyalkoxanoates, saccharide esters such as sorbitan monooleate, polysaccharide esters, free fatty acids, and fatty acid esters and salts. Other surfactants known to those skilled in the art may also be used.

TABLE 1
Exemplary porous material formulations

| Component | Amount (vol %) |
|---|---|
| Polymer | 40-85% |
| Ceramic | 0-40% |
| Fibers | 0-20% |
| Surfactant | 0-5% |

TABLE 2
Physical attributes of the porous material formulations of Table 1:

| | |
|---|---|
| Porosity | 30-90% |
| Average pore size | 10-500 μm |
| Compressive strength | 0.5-30 MPa |
| (parallel to fiber orientation) | |
| Time for complete | 6 weeks to 2 years |
| degradation | |

Any porous portions of the tissue spacer can be fabricated through polymer precipitation and vacuum expansion. Methods for the preparation of precipitated polymers are well-known to the art. In general, the process comprises mixing a dried polymer mix with a solvent, e.g. acetone, precipitating the polymer mass from solution with a non-solvent, e.g. ethanol, methanol, ether or water, extracting solvent and precipitating agent from the mass until it is a coherent mass which can be pressed into a mold or extruded into a mold, and curing the composition to the desired shape and stiffness. The optional surfactant is incorporated into the matrix of the material at the time of manufacture. Methods for incorporating reinforcement materials such as fibers and ceramics are known to the art. Methods for incorporating fiber reinforcements, for example, are described in U.S. Pat. No. 6,511,511, hereby incorporated by reference. Kneading and rolling may be performed as described in U.S. Pat. Nos. 6,511,511 and 6,203,573, hereby incorporated by reference. Curing and foaming the polymer in the mold to form a porous implant may then be done.

The material for the anterior plate may be an absorbable polymer optionally combined with a ceramic component and/or a buffering component. The anterior plate may also be made of a nonabsorbable material such as a polymer, a metal, or a ceramic. The anterior plate may be joined to the first region by making the first region and anterior plate as a single piece. Alternately, the anterior plate may be mechanically attached to the first region by screws, rivets, or snaps or other means as known in the art. The anterior plate may also be chemically bonded to the first region. The location of the connection between the plate and the load bearing portion may depend on the type of connection.

In another embodiment, the invention provides an orthopedic implant comprising
a tissue spacer having a superior and an inferior surface, the tissue spacer comprising
   a first region in the form of a ring, the ring having a superior surface which forms part of the superior surface of the spacer, an inferior surface which forms part of the inferior surface of the spacer, an inner surface and an outer surface; and
   a second region, the second region having a superior surface which forms part of the superior surface of the spacer, an inferior surface which forms part of the inferior surface of the spacer and an outer surface connected to the inner surface of the first region
wherein the porosity of the first region is between zero and about 30%, the porosity of the second region is between about 50% and about 90% and the second region comprises absorbable material.

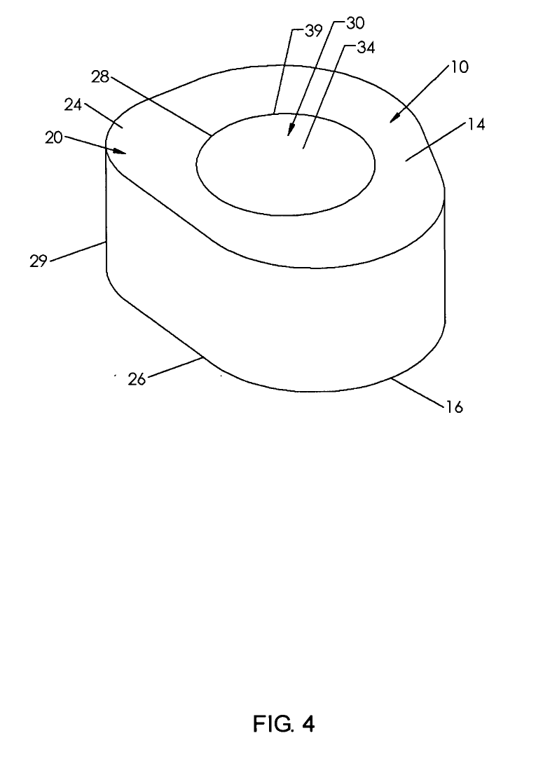

FIG. 4 illustrates a perspective view of a tissue spacer (10) comprising a less porous outer ring (20) and a more porous inner core (30) connected to the inner surface of the outer ring. The tissue spacer (10) has a superior surface (14) and an inferior surface (16). The ring (20) has a superior surface (24) an inferior surface (26), an inner surface (28) and an outer surface (29). The inner core (30) has a superior surface (34), an inferior surface (not shown), and an outer surface (39). The inner surface of ring (20) connects to the inner surface of ring (30). The outer ring can optionally be connected to an anterior plate.

The embodiment shown in FIG. 4 may be used as a spinal implant. When used as a spinal implant, the outer ring can be designed to substantially match the mechanical properties of cortical bone while the inner core can be designed to substantially match the mechanical properties of cancellous bone. The outer ring preferably has a Young's Modulus between about 1.0 GPa and about 30 GPa and a compressive strength between about 10 MPa and about 500 MPa. In an embodiment, the initial Young's Modulus is between about 10 GPa and about 30 GPa. The inner core preferably has a Young's modulus between about 0.5 GPa and about 5 GPa and a compressive strength between zero MPa and about 30 MPa. In an embodiment, the initial Young's Modulus is between about 1 GPa and about 5 GPa.

The primary function of the outer ring is to withstand high compressive loads. In an embodiment, the load-bearing outer ring is fully dense. In an embodiment, the load-bearing outer ring is formed of a substantially nonporous (fully dense) absorbable material comprising absorbable polymer, an optional ceramic component such as beta-tricalcium phosphate and an optional buffer such as calcium carbonate. This material was previously discussed as a suitable material for the first region of a different embodiment. Other nonabsorbable materials such as polymers, metals, or ceramics may be suitable for the outer ring. In other embodiments, the outer ring is not fully dense and has porosity less than about 40%, preferably less than about 35%. Absorbable polymers can be chosen, as is known to the art, to have a selected degradation period. For intervertebral spacers the degradation period is preferably up to about 4 years, or between about 6 weeks and about 2 years, or between about 12 weeks and about 1 year.

In an embodiment, the porosity of the inner core is between about 50% and about 90%. In different embodiments, the porosity of the full or partial wedge is greater than about 50% or greater than about 70%. Preferably, the inner core is sufficiently porous to allow for bony ingrowth. In an embodiment, the average pore size of the full or partial wedge between about 10 microns and about 2000 microns, between about 50 microns and about 900 microns and about 100 microns to about 600 microns. The more porous portion of the tissue spacer can be capable of soaking up fluids such as blood or bone marrow and therefore can be loaded with bioactive agents, drugs or pharmaceuticals. The inner core may be made of the same materials that are suitable for use in the full or partial wedge previously discussed. For the inner core, the degradation period is preferably up to about 2 years, or between about 3 weeks and about 1 year, or between about 6 weeks and about 9 months.

The embodiment shown in FIG. 4 can be fabricated by using the outer ring as a mold for the more porous inner core. Alternately, the outer and inner rings may be fabricated separately and then joined. Solvent can be used to adhere the inner core to the outer ring or other adhesives may be used as known in the art.

If an anterior plate is attached to the outer ring, the material for the anterior plate may be an absorbable polymer optionally combined with a ceramic component and/or a buffering component. The anterior plate may also be made of a nonabsorbable material such as a polymer, a metal, or a ceramic. The anterior plate may be joined to the plate by making the ring and anterior plate as a single piece. Alternately, the anterior plate may be mechanically attached to the ring by screws, rivets, or snaps or other means as known in the art. The anterior plate may also be chemically bonded to the ring.

In another embodiment, the invention provides an absorbable orthopedic implant comprising
a tissue spacer having a superior and an inferior surface, the tissue spacer comprising
   a ring, the ring having a superior surface which forms part of the superior surface of the spacer, an inferior surface which forms part of the inferior surface of the spacer, an inner and an outer surface; and
   an anterior plate connected to the ring.
wherein the ring has porosity between zero and about 15%.

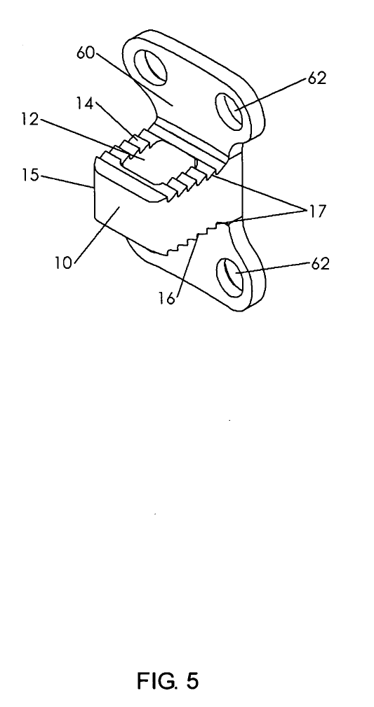

As illustrated in FIG. 5, the tissue spacer (10) comprises a ring (15) surrounding a void (12) running from the superior surface of the ring (14) to the inferior surface (16) of the ring. The superior and inferior surfaces of the ring can comprise teeth (17). The void can be left empty to be filled by materials selected by the surgeon, such as bone chips (auto or allograft) or can be fitted with a porous scaffold. The porous scaffold may be of the same material used for the inner core described previously. Alternately, commercially available porous scaffolds of different materials may be used. Fitting with a porous scaffold allows more efficient implementation of bioactive agents as compared to incorporation of bioactive agents into a fully dense material. FIG. 5 also shows the tissue spacer connected to an anterior plate (60) having holes (62) for use with screws.

The embodiment shown in FIG. 5 can be used as a spinal implant. When used as a spinal implant, the tissue spacer is placed into an interbody space where a discectomy was previously performed. The superior and inferior surfaces of the ring may comprise teeth to assist in retaining the tissue spacer in the interbody space. The anterior plate is designed to attach the anterior face of the adjacent vertebral bodies and may contain holes for screws. The anterior plate of the implant can be slightly contoured to match the natural curvature of the anterior vertebrae body faces. For some applications, such as cervical applications, an anterior plate may not be required.

The ring preferably has an initial Young's Modulus between about 1.0 GPa and about 30 GPa and a compressive strength between about 10 MPa and about 500 MPa. In an embodiment, the initial Young's Modulus is between about 10 GPa and about 30 GPa.

In an embodiment, the ring is formed of a substantially nonporous (fully dense) absorbable material comprising absorbable polymer, an optional ceramic component such as beta-tricalcium phosphate and an optional buffer such as calcium carbonate. This material was previously discussed as a suitable material for the first region of a different embodiment. Nonabsorbable polymers, metals or ceramics may also be used for the ring. Absorbable polymers can be chosen, as is known to the art, to have a selected degradation period. For intervertebral spacers, the degradation period is preferably up to about 4 years, or between about 6 weeks and about 2 years, or between about 12 weeks and about 1 year.

The material for the anterior plate may be an absorbable polymer optionally combined with a ceramic component and/or a buffering component. The anterior plate may also be made of a nonabsorbable material such as a polymer, a metal, or a ceramic. The anterior plate may be joined to the ring by making the ring and anterior plate as a single piece. Alternately, the anterior plate may be mechanically attached to the ring by screws, rivets, or snaps or other means as known in the art. The anterior plate may also be chemically bonded to the ring.

All references cited herein are hereby incorporated by reference to the extent that there is no inconsistency with the disclosure of this specification.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements. The invention illustratively described herein suitably may be practiced in the absence of any element or elements, limitation or limitations which is not specifically disclosed herein.

Whenever a range is given in the specification, for example, a time range or a composition range, all intermediate ranges and subranges, as well as all individual values included in the ranges are intended to be included in the disclosure.

Although the description herein contains many specificities, these should not be construed as limiting the scope of the invention, but as merely providing illustrations of some of the embodiments of the invention. Thus, additional embodiments are within the scope of the invention and within the following claims. The following examples are provided for illustrative purposes, and are not intended to limit the scope of the invention. Any variations in the materials, methods and devices herein which would occur to the skilled artisan from the inventive teachings herein are within the scope and spirit of the present invention.

### EXAMPLES

### Example 1

### Integrated Osteotomy Wedge

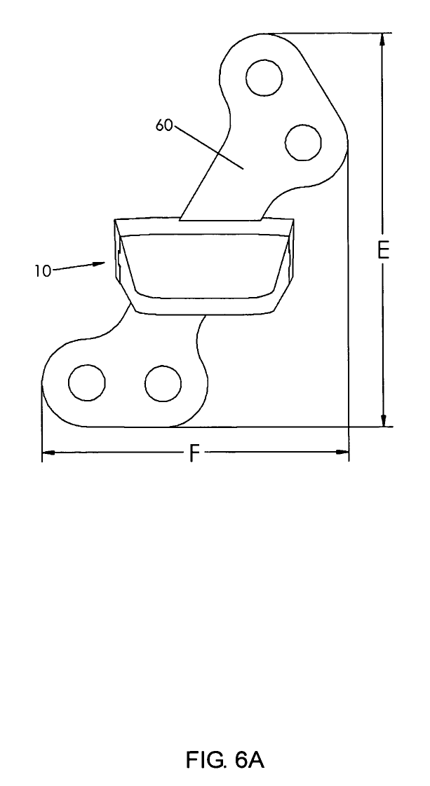

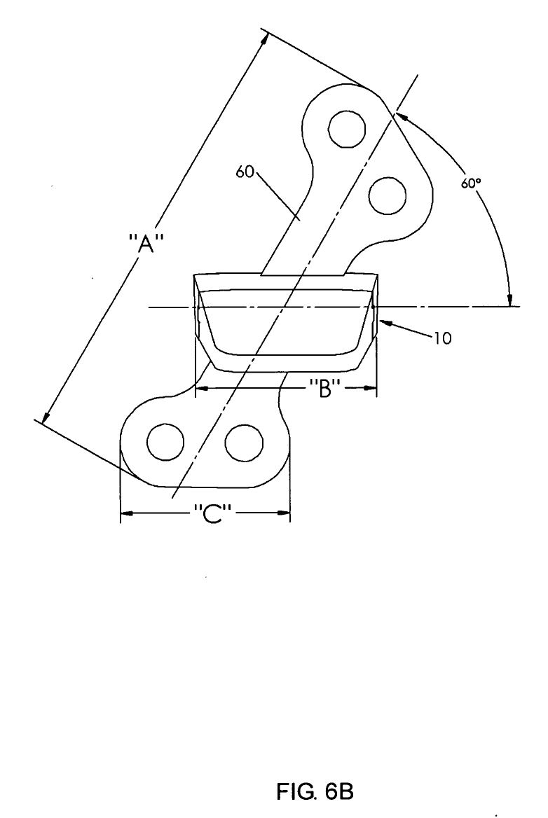

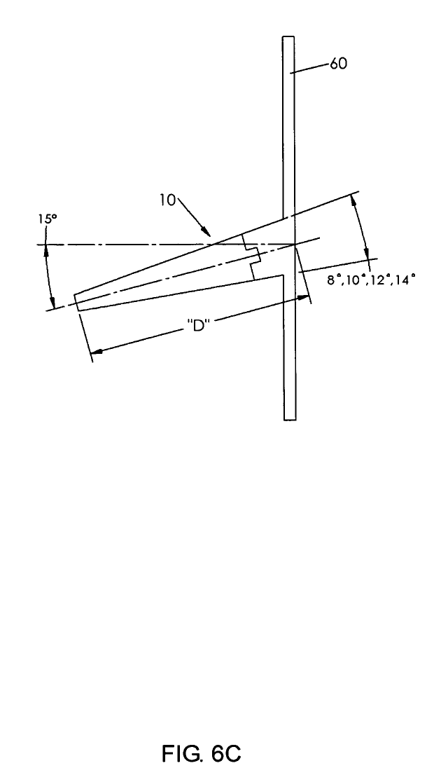

The implant shown in FIGS. 3A and 3B can be dimensioned for use as an osteotomy wedge for a high tibial implant. FIG. 6A illustrates dimensions of interest. FIG. 6A illustrates the distance, E, between the inferior and superior ends of the anterior plate, (60). The distance E is preferably between about 20 mm and about 130 mm. In an embodiment, the distance E is about 59 mm. FIG. 6A also illustrates the distance F between the left and right sides of the anterior plate. The distance F is preferably between about 20 mm and about 70 mm. In an embodiment, the distance F is about 46 mm. FIG. 6B illustrates distances A, B, and C. In an embodiment, the distance A is between about 25 mm and about 150 mm, the distance B between about 5 mm and about 50 mm, and the distance C between about 10 mm and about 50 mm. FIG. 6C illustrates distance D. In an embodiment, distance D is between about 20 mm and about 70 mm.

### Example 2

### Integrated Cervical Spacer

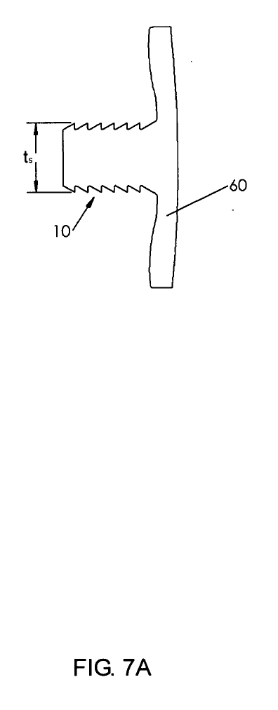

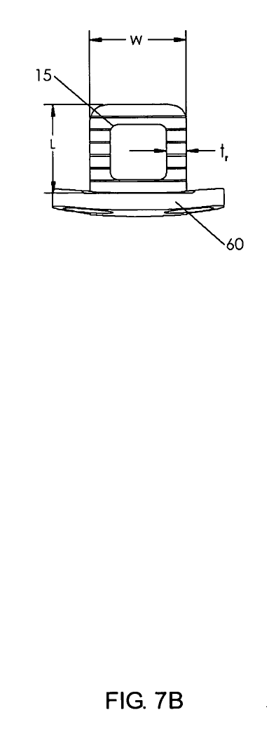

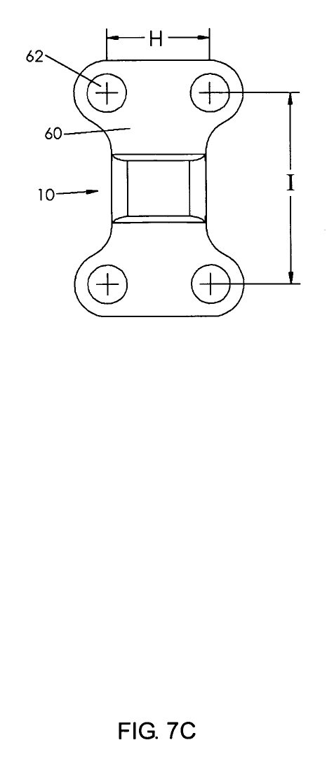

The implant shown in FIG. 5 can be dimensioned for use as an integrated cervical spacer. FIGS. 7A-7C illustrate dimensions of interest. FIG. 7A illustrates the thickness, tₛ, of the interbody spacer (10). Preferred thicknesses for the interbody spacer are between about 5 mm and about 50 mm. FIG. 7B illustrates an interbody spacer having a square ring (15) with length, L, width, w, and thickness tr. In an embodiment, L is between about 8 mm and about 15 mm and w is between about 8 mm and about 16 mm. In an embodiment, the length and width are both equal to about 11 mm. Preferred thicknesses for the ring are between about 0.5 mm and about 3 mm. In an embodiment, the ring thickness is about 2.5 mm. FIG. 7C illustrates the horizontal spacing, H, between the holes (62) in the anterior plate (60). The preferred horizontal spacing is between about 10 mm and about 25 mm. In an embodiment, the horizontal spacing is about 12 mm. FIG. 7C also illustrates the vertical spacing, H, between the holes (62) in the anterior plate. The preferred vertical spacing is between about 15 mm and about 100 mm. In an embodiment, the vertical spacing is about 22 mm.

### Example 3

### A Porous Composition Useful for Filling Bony Defects

| Component | Type | Amount (vol %) |
|---|---|---|
| Polymer | 85/15 DL-PLG | 54.50% |
| Ceramic | Calcium sulfate dihydrate | 35.00% |
| Fibers | PGA chopped fibers | 9.00% |
| Surfactant | Pluronic F127NF | 1.50% |

The material is formed into a 75% porous construct, average pore size 200 μm, with an initial compressive strength of 2.4 MPa and compressive stiffness of >100 MPa. The calcium sulfate fraction dissolves in about 4-6 weeks and the polymer fraction dissolves in about 8 months. The material can be fabricated into plugs, blocks, cubes, and granules to fill a wide variety of defects.

### Example 4

### A Porous Composition Useful for Supporting Load-Bearing Bony Defects

| Component | Type | Amount (vol %) |
|---|---|---|
| Polymer | 70/30 L/DL-PLA | 58.50% |
| Ceramic | HydroxyGPatite | 25.00% |
| Fibers | L-PLA chopped fibers | 15.00% |
| Surfactant | Pluronic F127NF | 1.50% |

The material is formed into a 70% porous construct with an initial compressive strength of 16 MPa and compressive stiffness of >200 MPa. The hydroxyapatite fraction absorbs slowly by osteoclastic activity, and the polymer fraction dissolves in about 18-36 months.

### Example 5

### Nonporous Absorbable Composite Compositions

Nonporous absorbable materials may comprise polylactic acid, beta phase tricalcium phosphate and calcium carbonate. Exemplary compositions are summarized in Table 3. The nonporous materials can be made by dry blending the polymer resin with the ceramic components prior to injection molding. The particle size can range from about 10 to about 70 microns. In an embodiment, the particle size is about 20 to about 40 microns.

TABLE 3
Material Compositions of Fully Dense Absorbable Composites

| | Matrix | | βTCP | CaCO₃ |
|---|---|---|---|---|
| | Polymer | Weight % | Weight % | Weight % |
| Composite 1 | 70/30 P(L, D/L)LA | 80.0 | 16.0 | 4.0 |
| (C1) | | | | |
| Composite 2 | 70/30 P(L, D/L)LA | 62.0 | 31.0 | 7.0 |
| (C2) | | | | |
| Composite 3 | PLLA | 62.0 | 31.0 | 7.0 |
| (C3) | | | | |

### Example 6

### Mechanical Testing of Nonporous Absorbable Composites

Several compositions of nonporous absorbable composites were tested to determine their mechanical properties. 3.5 mm diameter rods (length=70 mm) were used for flexural testing and 5.5 mm rods (cut to ˜5.5 mm in length) were used for compression testing. For comparison, at approximately room temperature unreinforced poly(L-lactide) has a compressive strength of 125.5 MPa and a compressive modulus of 5.1 GPa (Verheyen, C C et. al. “Evaluation of hydroxlapatite/poly(L-lactide) composites: Mechanical behavior” J of Biomed Mat Res, Vol 26, 1277-1296 (1992)). Unreinforced poly(D,L) lactide has a bending strength of 101.6 MPa and a bending modulus of 2.25 GPa (Heidemann, W et. al. “Degradation of poly(D,L)lactide implants with or without addition of calciumphosphates in vivo” Biomaterials, Vol 22, 2371-2381 (2001)).

The accelerated degradation study used a buffered simulated body fluid (pH=7.4) at 47° C. At the appropriate evaluation time points, the samples were removed from the 47° C. incubator and were preconditioned at 37° C. for at least one hour prior to testing. Flexural samples were tested in wet conditions at 37° C. in 3-point bend per ASTM D-790. Samples tested in compression were removed from the buffered solution and placed in vials. These samples were sent overnight to a contract testing lab. Prior to testing, compression samples were preconditioned in deionized water for at least two (2) hours. The test temperature was 37° C. Compression testing was conducted on Composite 2 and Composite 3 using a loading rate of 6 mm/min. The testing was stopped after the initial yield stress was visualized.

Tables 4 and 5 summarize the mechanical properties of three composites that were degraded at 47° C.

TABLE 4
Mechanical Strength Values for Absorbable Composite Materials

| | | | | Compressive Strength | |
|---|---|---|---|---|---|
| | Time | Flexural Strength (MPa) | | (MPa) | |
| Sample | (weeks) | Average | Std Dev. | Average | Std Dev. |
| Composite 1 | 0 | 76.53 | 1.12 | — | — |
| | 1 | 43.99 | 6.43 | — | — |
| | 2 | 14.73 | 5.86 | — | — |
| | 3 | 1.92 | 1.44 | — | — |
| | 4 | — | — | — | — |
| | 5 | — | — | — | — |
| | 6 | — | — | — | — |
| Composite 2 | 0 | 66.70 | 3.75 | 106.33 | 6.57 |
| | 1 | 36.92 | 3.38 | 85.90 | 7.59 |
| | 2 | 21.73 | 15.64 | 69.48 | 6.24 |
| | 3 | 19.28 | 3.11 | 59.42 | 8.03 |
| | 4 | 4.07 | 6.23 | 48.64 | 4.64 |
| | 5 | — | — | 42.90 | 4.26 |
| | 6 | — | — | 33.96 | 4.13 |
| Composite 3 | 0 | 74.90 | 3.46 | 112.60 | 3.08 |
| | 1 | 45.26 | 2.11 | 97.35 | 3.28 |
| | 2 | 42.85 | 1.19 | 91.60 | 4.44 |
| | 3 | 42.28 | 4.16 | 83.85 | 4.62 |
| | 4 | 35.29 | 2.72 | 83.59 | 2.72 |
| | 5 | 37.82 | 6.08 | 76.35 | 4.57 |
| | 6 | 28.13 | 4.12 | 75.23 | 6.43 |

TABLE 5
Mechanical Stiffness Values for Absorbable Composite Materials

| | | | | Compressive Modulus | |
|---|---|---|---|---|---|
| | Time | Flexural Modulus (MPa) | | (MPa) | |
| Sample | (weeks) | Average | Std Dev. | Average | Std Dev. |
| Composite 1 | 0 | 4017.81 | 69.22 | — | — |
| | 1 | 2559.63 | 77.12 | — | — |
| | 2 | 1084.77 | 207.06 | — | — |
| | 3 | — | — | — | — |
| | 4 | — | — | — | — |
| | 5 | — | — | — | — |
| | 6 | — | — | — | — |
| Composite 2 | 0 | 4629.93 | 178.87 | 2678.75 | 219.89 |
| | 1 | 2448.57 | 186.39 | 1973.40 | 389.08 |
| | 2 | 2422.63 | 367.22 | 1320.20 | 237.18 |
| | 3 | 1251.86 | 218.98 | 1210.20 | 288.19 |
| | 4 | — | — | 916.00 | 171.51 |
| | 5 | — | — | 826.80 | 162.49 |
| | 6 | — | — | 330.60 | 77.78 |
| Composite 3 | 0 | 5069.80 | 347.09 | 2787.27 | 631.86 |
| | 1 | 2686.29 | 167.68 | 2028.85 | 288.23 |
| | 2 | 2558.29 | 108.68 | 1823.14 | 135.67 |
| | 3 | 2386.23 | 350.20 | 1624.52 | 105.97 |
| | 4 | 2118.57 | 166.94 | 1652.41 | 66.86 |
| | 5 | 2165.62 | 296.26 | 1433.23 | 169.18 |
| | 6 | 1589.68 | 89.03 | 1470.67 | 226.89 |

A real-time degradation study used a buffered simulated body fluid (pH=7.4) at 37° C. to determine change in compressive properties of a 70/30 PLDLA composite. The results are shown in Table 6.

TABLE 6
Real Time Degradation Compressive Properties for a 70/30 PLDLA
Composite

| | |
|---|---|
| Compressive Strength | Compressive Modulus |
| (MPa) | (MPa) |

| Time Period | Average | St Dev | Average | St Dev |
|---|---|---|---|---|
| Time 0 | 107.03 | 4.42 | 2894.95 | 102.13 |
| Week 1 | 77.97 | 3.76 | 1339.89 | 43.09 |
| Week 4 | 80.18 | 10.36 | 1758.60 | 355.01 |
| week 8 | 68.03 | 10.37 | 1400.74 | 450.3 |
| Week 12 | 66.46 | 5.42 | 822.59 | 172.33 |
| Week 15 | 73.02 | 11.53 | 1225.16 | 305.12 |
| Week 24 | 52.35 | 11.19 | 685.77 | 181.06 |

1. An orthopedic implant comprising
a tissue spacer having a superior and an inferior surface, the tissue spacer comprising
a first region having a superior surface which forms part of the superior surface of the spacer, an inferior surface which forms part of the inferior surface of the spacer, an inner surface and an outer surface; and
a second region in the form of a full or partial wedge, the second region having a superior surface which forms part of the superior surface of the spacer, an inferior surface which forms part of the inferior surface of the spacer, and an anterior surface connected to the inner surface of the first region;
wherein the second region is more porous than the first region and comprises absorbable material and the first region is not in the form of a complete ring.

2. The implant of claim 1 wherein the porosity of the first region is between zero and about 30%.

3. The implant of claim 1 wherein the porosity of the first region is between zero and about 15%.

4. The implant of claim 1 wherein the first region is substantially nonporous.

5. The implant of claim 1 wherein the porosity of the second region is between about 50% and about 90%.

6. The implant of claim 1 wherein the porosity of the second region is between about 70% and about 90%.

7. The implant of claim 1 wherein the first region comprises an absorbable polymer.

8. The implant of claim 7 wherein the first region additionally comprises ceramic particles.

9. The implant of claim 8 wherein the ceramic particles are beta-tricalcium phosphate particles.

10. The implant of claim 7 wherein the first region additionally comprises a buffer.

11. The implant of claim 10 wherein the buffer is calcium carbonate.

12. The implant of claim 1 wherein the second region comprises a porous composite of an absorbable polymer, ceramic particles, fibers, and surfactant.

13. The implant of claim 1 wherein the implant additionally comprises an anterior plate connected to the first region.

14. The implant of claim 13 wherein the angle between the midplane of the tissue spacer and the anterior plate is between about 55 degrees and about 90 degrees.

15. The implant of claim 13 wherein the angle between the midplane of the tissue spacer and the midplane of the anterior plate is between about 30 degrees and about 90 degrees.

16. The implant of claim 13 wherein the anterior plate additionally comprises at least one hole which can be used for attachment of the plate to a bone.

17. The implant of claim 13 wherein the anterior plate comprises an absorbable polymer.

18. The implant of claim 1, wherein the second region is loaded with a bioactive agent, drug, pharmaceutical agent, cells or combinations thereof.

19. An orthopedic implant comprising
a tissue spacer having a superior and an inferior surface, the tissue spacer comprising
a first region in the form of a ring, the ring having a superior surface which forms part of the superior surface of the spacer, an inferior surface which forms part of the inferior surface of the spacer, an inner surface and an outer surface; and
a second region, the second region having a superior surface which forms part of the superior surface of the spacer, an inferior surface which forms part of the inferior surface of the spacer and an outer surface connected to the inner surface of the first region;
wherein the porosity of the first region is between zero and about 30%, the porosity of the second region is between about 50% and about 90%, and the second region comprises absorbable material.

20. The implant of claim 19 wherein the porosity of the first region is between zero and about 15%.

21. The implant of claim 19 wherein the first region is substantially nonporous.

22. The implant of claim 19 wherein the porosity of the second region is between about 70% and about 90%.

23. The implant of claim 19 wherein the first region comprises an absorbable polymer.

24. The implant of claim 23 wherein the first region additionally comprises ceramic particles.

25. The implant of claim 24 wherein the ceramic particles are beta-tricalcium phosphate particles.

26. The implant of claim 23 wherein the first region additionally comprises a buffer.

27. The implant of claim 26 wherein the buffer is calcium carbonate.

28. The implant of claim 19 wherein the second region comprises a porous composite of an absorbable polymer, a ceramic, fibers, and surfactant.

29. The implant of claim 19 wherein the implant additionally comprises an anterior plate connected to the ring.

30. The implant of claim 29, wherein the anterior plate additionally comprises at least one hole which can be used for attachment of the plate to a bone.

31. The implant of claim 29 wherein the anterior plate comprises an absorbable polymer.

32. The implant of claim 19 wherein the second region is loaded with a bioactive agent, drug, pharmaceutical agent, cells, or combinations thereof.

33. An absorbable orthopedic implant comprising
a tissue spacer having a superior and an inferior surface, the tissue spacer comprising
a ring, the ring having a superior surface which forms part of the superior surface of the spacer, an inferior surface which forms part of the inferior surface of the spacer, an inner and an outer surface; and
an anterior plate connected to the ring
wherein the ring has porosity between zero and about 15%.

34. The implant of claim 33 wherein the ring comprises an absorbable polymer.

35. The implant of claim 34 wherein the ring additionally comprises ceramic particles.

36. The implant of claim 35 wherein the ceramic particles are beta-tricalcium phosphate particles.

37. The implant of claim 34 wherein the ring additionally comprises a buffer.

38. The implant of claim 37 wherein the buffer is calcium carbonate.
